(19) United States
(12) Patent Application Publication
(10) Pub. No.: US 2018/0055761 A1
(43) Pub. Date: Mar. 1, 2018

(54) TOPICAL PRODUCT INCLUDING APPLE

(71) Applicant: Ross Anapolle, Newton, MA (US)

(72) Inventor: Ross Anapolle, Newton, MA (US)

(21) Appl. No.: 15/693,897

(22) Filed: Sep. 1, 2017

Related U.S. Application Data
(60) Provisional application No. 62/382,375, filed on Sep. 1, 2016.
(60) Provisional application No. 62/382,399, filed on Sep. 1, 2016.

(51) Int. Cl. A61K 8/9789; A61Q 19/00; A61K 36/73

(52) U.S. Cl. CPC A61K 8/9789; A61K 36/73; A61Q 19/007

(57) ABSTRACT

Provided is a topical apple product, comprising a base topical product configured for application to a human and an apple material substantially uniformly combined with the base topical material. Also provided is a method of treating a skin condition or ailment with topical apple product. The apple material can have a Polyphenols/Total Antioxidant Activity of ≧3.00%.

### CROSS REFERENCE TO RELATED APPLICATIONS

This application claims the benefit of priority under 35 U.S.C. §119(e) from provisional application Ser. No. 62/382,375, entitled TOPICAL PRODUCT INCLUDING DRIED APPLE PEEL POWDER, filed on Sep. 1, 2016, and provisional application Ser. No. 62/382,399, entitled TOPICAL PRODUCT INCLUDING APPLE PEEL EXTRACT, filed on Sep. 1, 2016, which are incorporated herein by reference in their entireties.

### FIELD OF INTEREST

The present inventive concepts relate to the field of topical products, in particular, the present invention relates to topical products comprising apple, apple constituents, and/or apple byproducts, such as apple peel powder, apple extract, and the like.

### SUMMARY

In accordance with aspects of the present disclosure, provided is a topical product comprising an apple material, sometimes referred to herein as a “topical apple product.” The apple material can take the form of an apple peel powder, liquid extract, or the like. The apple material can comprise apple peel, apple, flesh and/or combinations thereof. The topical apple product can comprise the apple material combined with a base topical product. The topical apple product can take the form of a liquid, lotion, gel, paste, salve, ointment, cream, rub, powder, extract, wax, or spray, as examples, that can be applied to skin, hair, teeth, and/or nails.

In accordance with aspects of the present disclosure, provided is a topical product, comprising a base topical product configured for application to a human and an apple material substantially uniformly combined with the base topical material.

In various embodiments, the apple material comprises dried apple peel powder.

In various embodiments, the apple material comprises liquid apple extract.

In various embodiments, the apple material comprises apple peel.

In various embodiments, the apple material comprises apple flesh.

In various embodiments, the apple material comprises antioxidants.

In various embodiments, the apple material comprises Polyphenols/Total Antioxidant Activity of ≧3.00%.

In various embodiments, the apple peel extract comprises Polyphenols/Total Antioxidant Activity in a range of about 20% to about 35%.

In various embodiments, the base topical product is a cosmetic product.

In various embodiments, the cosmetic product is chosen from a group consisting of: face and/or body lotion, such as moisturizers and cleansers, skin serums, body powder, sunscreen, bronzer, shampoo, conditioner, soap, hair color, hair detangler, mask, peel, lipstick, lip gloss, lip balm, foundation, blush, toner, mascara, eye shadow, eye and/or anti-aging solutions, shave cream, lotion, or gel, aftershave, deodorant, antiperspirant, cologne, perfume, body splash, toothpaste, floss, and/or mouthwash and/or mouth rinse.

In various embodiments, the base topical material is chosen from a group consisting of: medical treatment product, such as a first aid cream or spray or other skin treatment cream or spray, such as for use to treat a wound, skin irritation, burn, rash, eczema, infection, and/or melanoma, or the like.

In various embodiments, the topical apple product is suitable for application to human skin.

In various embodiments, the topical apple product is suitable for application to human hair.

In various embodiments, the topical apple product is suitable for application to human teeth.

In various embodiments, a percentage of apple material in the topical apple product is greater than 0% and up to about 50%.

In various embodiments, the percentage of apple material in the topical apple product is greater than 0% and up to about 25%.

In various embodiments, the percentage of apple material in the topical apple product is greater than 0% and up to about 10%.

In accordance with another aspect of the inventive concept, provided is a topical applicant product, comprising a base topical product configured for application to a human and an apple material substantially uniformly combined with the base topical material. The apple material is a dried apple peel powder having Polyphenols/Total Antioxidant Activity of ≧3.00%, the apple peel extract topical is suitable for application to human skin, and the base topical product is chosen from a group consisting of: face and/or body lotion, such as moisturizers and cleansers, skin serums, body powder, sunscreen, bronzer, shampoo, conditioner, soap, hair color, hair detangler, mask, peel, lipstick, lip gloss, lip balm, foundation, blush, toner, mascara, eye shadow, eye and/or anti-aging solutions, shave cream, lotion, or gel, aftershave, deodorant, antiperspirant, cologne, perfume, body splash, toothpaste, floss, and/or mouthwash and/or mouth rinse.

In accordance with another aspect of the inventive concept, provided is a method of treating a skin condition and/or ailment, including applying a topical apple product to a portion of skin condition repair skin dryness, irritation, rash, and/or melanoma.

In various embodiment, the apple material has a Polyphenols/Total Antioxidant Activity of ≧3.00%.

In accordance with aspects of the present disclosure, provided is a method of making a topical apple product, comprising providing a base topical product configured for application to a human and combining an apple material with the topical product.

### BRIEF DESCRIPTION OF THE DRAWINGS

The present invention will become more apparent in view of the attached drawings and accompanying detailed description. The embodiments depicted therein are provided by way of example, not by way of limitation, wherein like reference numerals refer to the same or similar elements. The drawings are not necessarily to scale, emphasis instead being placed upon illustrating aspects of the invention. In the drawings:

FIG. 1 is a front view of a container partially filled with a base topical product, in accordance with the prior art;

FIG. 2 is a front view of an embodiment of a container partially filled with an apple material, in accordance with aspects of the inventive concept;

FIG. 3 is a notional view of an embodiment of a process for making a topical apple product, in accordance with aspects of the inventive concept;

FIG. 4 is a flowchart showing an embodiment of a method of making a topical apple product, in accordance with aspects of the inventive concept; and

FIG. 5 is a flowchart showing an embodiment of another method of making a topical apple product, in accordance with aspects of the inventive concept.

### DETAILED DESCRIPTION OF PREFERRED EMBODIMENTS

Various aspects of the inventive concepts will be described more fully hereinafter with reference to the accompanying drawings, in which some exemplary embodiments are shown. The present inventive concept may, however, be embodied in many different forms and should not be construed as limited to the exemplary embodiments set forth herein.

It will be understood that, although the terms first, second, etc. are be used herein to describe various elements, these elements should not be limited by these terms. These terms are used to distinguish one element from another, but not to imply a required sequence of elements. For example, a first element can be termed a second element, and, similarly, a second element can be termed a first element, without departing from the scope of the present invention. As used herein, the term “and/or” includes any and all combinations of one or more of the associated listed items.

It will be understood that when an element is referred to as being “on” or “connected” or “coupled” to another element, it can be directly on or connected or coupled to the other element or intervening elements can be present. In contrast, when an element is referred to as being “directly on” or “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, steps, operations, elements, and/or components, but do not preclude the presence or addition of one or more other features, steps, operations, elements, components, and/or groups thereof.

In accordance with aspects of the present invention, there are provided various topical apple products comprising apple material, such as apple peel powder, extract or the like. As will be discussed further below, the topical apple products can take any of a variety of forms, including, but not limited to, cosmetic, and non-cosmetic topically applied to the skin, hair, teeth, or other tissue. Various forms of apple material have antioxidant properties that can be particularly advantageous for treating and/or repairing various skin ailments and/or conditions. The topical apple product can take the form of a liquid, lotion, gel, paste, salve, ointment, cream, rub, powder, extract, wax, or spray, as examples, that can be applied to skin, hair, teeth, and/or nails.

As an example, various methods have been used to make apple peel powder from apple skins, such as those described in U.S. Pat. No. 8,551,554 (the '554 patent”), the contents of which are incorporated herein by reference. The '554 patent primarily described manners of making such a powder in view of studies showing in vitro cancer fighting benefits. For therapeutic purposes, the '554 patent described making the apple peel powder to have an unusually high phenolic content, i.e., 3.00%. To the limited extent that uses of such a powder were disclosed in the '554 patent, they were limited to treating and inhibiting the proliferation of liver cancer cells. The apple peel powder is described as being made for oral consumption as a dietary supplement, in tablet or capsule form, or as a food additive or ingredient.

Leahy Orchards Inc. of Canada produces an organic dried apple peel powder under the tradename AppleActiv DAPP™. Some of the significant characteristics of AppleActiv DAPP™ are: high phenolic and flavonoid content, rich in antioxidants, enzymes, vitamins and minerals; and provide clinically supported health benefits. As in the '554 patent, this product is promoted as a dietary supplement or food ingredient to be consumed and digested by a living being (e.g., person). Other approaches to producing dried apple peel powder may also suffice, so the present invention need not be limited to the methods disclosed in the '554 patent.

In contrast to uses where apple peel powder (APP) is consumed and digested into the body, the present invention includes novel products and uses of products that include apple material, such as apple peel powder or extract, for application as a topical agent or ingredient in a topical apple product. The apple material can be an apple peel powder made for a method described in the '554 patent and/or can take the form of AppleActiv DAPP from Leahy Orchards Inc., or the like.

In a preferred form, the apple material preferably includes Polyphenols/Total Antioxidant Activity of ≧3.00%. But in some embodiments, a topical apple product having apple material having Polyphenols/Total Antioxidant Activity of 0.00%< and <3.00% could also provide treatment, repair, and/or prevention improvements and advantages over the base topical product without the apple material, e.g., a topical apple product having Polyphenols/Total Antioxidant Activity of 0.00%< and ≦0.50%, 0.50%< and ≦1.00%, 1.00%< and ≦1.50%, 1.50%< and ≦2.00%, 2.00%< and ≦2.50%, and/or 2.50%< and ≦3.00% could be advantageously provided. In some embodiments, the Polyphenols/Total Antioxidant Activity of can be between in a range of about 3% to about 10%, 10% to 20%, and/or 20% to about 35%, or more.

The topical apple product can be formed by adding the apple peel material to a “base product” or “base topical product.” The base product can take the form of a liquid, lotion, gel, paste, salve, ointment, cream, rub, powder, extract, wax, or spray, as examples. The base product could be a product suitable for application to skin, hair, teeth, and/or nails.

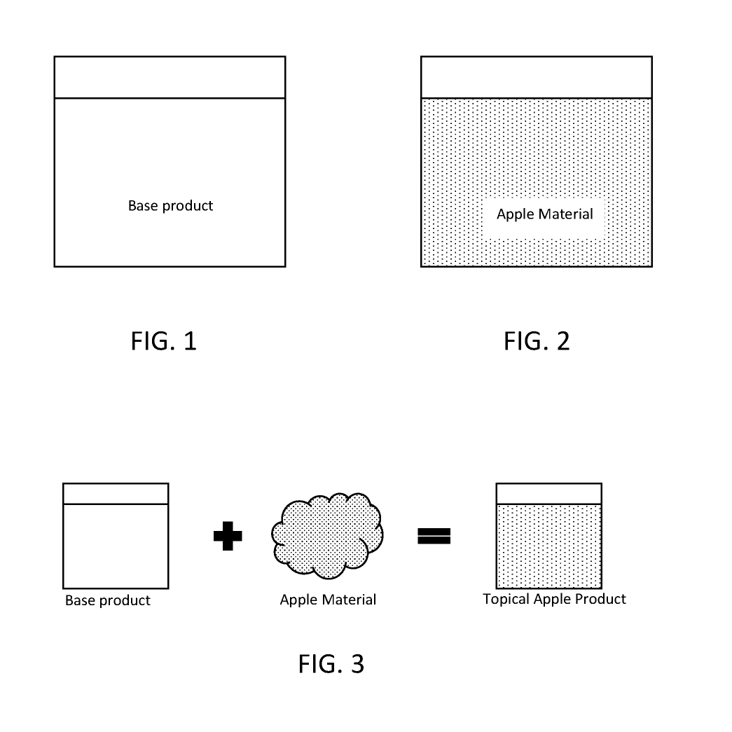

FIG. 1 shows an embodiment of a container holding a base topical product and FIG. 2 shows an embodiment of a container holding an apple material. The precise amount of apple material, e.g., dried apple peel powder or liquid extract added to a base topical product will vary depending on the intended use of the product. For example, when used in a cosmetic product, such as makeup, the apple material can be generally undetectable from the look, feel, consistency, and application perspectives of the base topical product. That is, in various embodiment the topical apple product can appear to the consumer and user to be no different, or not substantially different, than the base topical product without the apple material. The apple material need not alter, or substantially alter, the look, feel, consistency, and/or application of the base topical product, while providing significant repair and/or treatment benefits to the area of application (e.g., damaged skin).

As examples, the topical apple product can take the form of any of the following (base product+apple material (e.g., Dried apple peel powder, liquid apple extract, or the like): face and/or body lotion, such as moisturizers and cleansers, skin serums, body powder, sunscreen, bronzer, shampoo, conditioner, soap, hair color, hair detangler, mask, peel, lipstick, lip gloss, lip balm, foundation, blush, toner, mascara, eye shadow, eye and anti-aging solutions, shave cream, lotion, or gel, aftershave, deodorant, antiperspirant, cologne, perfume, body splash, toothpaste, floss, and/or mouthwash or mouth rinse. As other examples, the topical apple product can be a medical treatment product, such as a first aid cream or spray or other skin treatment cream or spray, such as for use to treat a wound, skin irritation, burn, rash, eczema, infection, and/or melanoma, or the like.

FIG. 3 shows a notional example of a base product and apple material being combined to form a topical apple product.

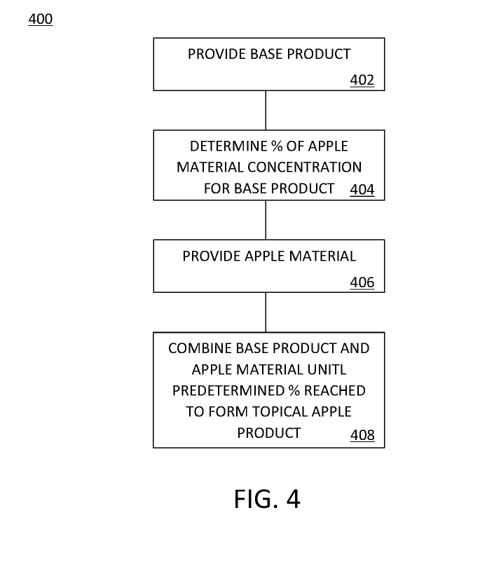

Depending on the particular base product, different techniques can be used to combine in the apple material with the base product. Preferably, in most embodiments, the selected technique achieves equal, or substantially equal, suspension and a uniform consistency in the base products, e.g., liquids, gels, or solids (e.g., lipstick, deodorant, etc.). That is, preferably, the addition of the apple material does not adversely alter the consistency of the base product. For example, the apple material, e.g., dried apple peel powder, liquid extract, or the like, can be combined (e.g., mixed) into a complete base product, such as a lotion, powder, gel, or the like, as shown in the representative method 400 of FIG. 4. Or, in other embodiments, the apple material, e.g., dried apple peel powder, liquid extract, or the like, can be combined (e.g., mixed) with base product constituents, during the process of making the base product to form a topical apple product.

In the method 400 of FIG. 4, a base topical product is provided in step 402. A percentage of applicant material within the final topical apple product is determined. The apple material is provided to achieve the determined percentage. And the base topical product and the apple material are combined to form the topical apple product having the predetermined amount of apple material.

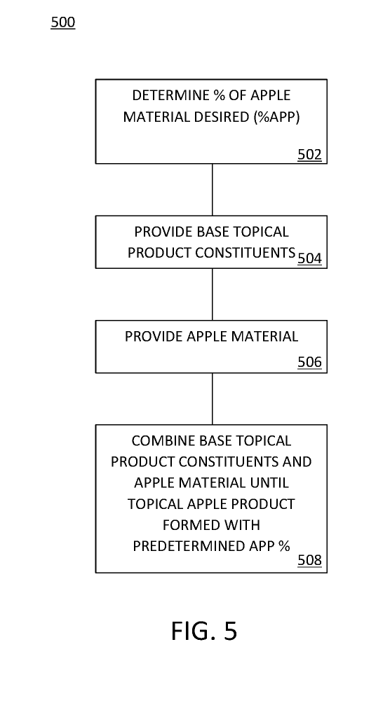

FIG. 5 shows another method 500 of making the topical apple product. In this method the apple material is added while making the base topical product, to collectively form the topical apple product. In step 502, a percentage of apple material is determined for a total amount of topical apple product or determined amount of base topical product. The base topical product constituents are provided and the apple material is provided as an additional constituents. The constituents are combined and processed to form a topical base product having the predetermined percentage of apple material, e.g., dried apple peel powder and/or liquid apple extract.

In various embodiments of methods used in making a topical apple product, an appropriate amount of apple material, e.g., as a percentage of the base product, can be predetermined, such as by a machine capable of volume and/or weight measures. This percentage can be variously determined based on several factors, such as not exceeding an amount (apple material threshold amount) that would adversely affect the look, feel, texture, consistency, odor, and/or performance of the base product, such as for cosmetics and other product that are not primarily palliative. For medical topical base products, some or all of these factors may be less of a consideration. For example, in various embodiments, a topical apple product may include a percentage of apple material that is greater than 0% and up to about 50%, while in other embodiments, the percentage may be greater than 0% and up to about 25%, while in still other embodiments, the percentage may be greater than 0% and up to about 10%, while still in other embodiments, the percentage may be greater than 0% and up to about 5%. Depending on the embodiment, the percentage of apple material in a topical apple product could be of still another percentage. In the methods of FIGS. 4 and 5, as examples, the amount of apple material to be added to the base product is predetermined. This can be done by an automated machine configured to determine the appropriate quantities of apple material and/or base topical material to form the topical apple product having the predetermined and desired amount of apple material.

While the foregoing has described what are considered to be the best mode and/or other preferred embodiments, it is understood that various modifications can be made therein and that the invention or inventions may be implemented in various forms and embodiments, and that they may be applied in numerous applications, only some of which have been described herein. It is intended by the following claims to claim that which is literally described and all equivalents thereto, including all modifications and variations that fall within the scope of each claim.

It is appreciated that certain features of the invention, which are, for clarity, described in the context of separate embodiments, may also be provide in combination in a single embodiment. Conversely, various features of the invention which are, for brevity, described in the context of a single embodiment may also be provided separately or in any suitable sub-combination.

For example, it will be appreciated that all of the features set out in any of the claims (whether independent or dependent) can be combined in any given way.

What is claimed is:

1. A topical product, comprising:
a base topical product configured for application to a human; and
an apple material substantially uniformly combined with the base topical material.

2. The product of claim 1, wherein the apple material comprises dried apple peel powder.

3. The product of claim 1, wherein the apple material comprises liquid apple extract.

4. The product of claim 1, wherein the apple material comprises apple peel.

5. The product of claim 1, wherein the apple material comprises apple flesh.

6. The product of claim 1, wherein the apple material comprises antioxidants.

7. The product of claim 1, wherein the apple material comprises Polyphenols/Total Antioxidant Activity of ≧3.00%.

8. The product of claim 1, wherein the apple peel extract comprises Polyphenols/Total Antioxidant Activity in a range of about 20% to about 35%.

9. The product of claim 1, wherein the base topical product is a cosmetic product.

10. The product of claim 9, wherein the cosmetic product is chosen from a group consisting of:
face and/or body lotion, such as moisturizers and cleansers, skin serums, body powder, sunscreen, bronzer, shampoo, conditioner, soap, hair color, hair detangler, mask, peel, lipstick, lip gloss, lip balm, foundation, blush, toner, mascara, eye shadow, eye and/or anti-aging solutions, shave cream, lotion, or gel, aftershave, deodorant, antiperspirant, cologne, perfume, body splash, toothpaste, floss, and/or mouthwash and/or mouth rinse.

11. The product of claim 1, wherein the base topical material is chosen from a group consisting of:
medical treatment product, such as a first aid cream or spray or other skin treatment cream or spray, such as for use to treat a wound, skin irritation, burn, rash, eczema, infection, and/or melanoma, or the like.

12. The product of claim 1, wherein the topical apple product is suitable for application to human skin.

13. The product of claim 1, wherein the topical apple product is suitable for application to human hair.

14. The product of claim 1, wherein the topical apple product is suitable for application to human teeth.

15. The product of claim 1, wherein a percentage of apple material in the topical apple product is greater than 0% and up to about 50%.

16. The product of claim 15, wherein the percentage of apple material in the topical apple product is greater than 0% and up to about 25%.

17. The product of claim 16, wherein the percentage of apple material in the topical apple product is greater than 0% and up to about 10%.

18. A topical applicant product, comprising:
a base topical product configured for application to a human; and
an apple material substantially uniformly combined with the base topical material,
wherein the apple material is a dried apple peel powder having Polyphenols/Total Antioxidant Activity of ≧3.00%,
wherein the apple peel extract topical is suitable for application to human skin, and
wherein the base topical product is chosen from a group consisting of:
face and/or body lotion, such as moisturizers and cleansers, skin serums, body powder, sunscreen, bronzer, shampoo, conditioner, soap, hair color, hair detangler, mask, peel, lipstick, lip gloss, lip balm, foundation, blush, toner, mascara, eye shadow, eye and/or anti-aging solutions, shave cream, lotion, or gel, aftershave, deodorant, antiperspirant, cologne, perfume, body splash, toothpaste, floss, and/or mouthwash and/or mouth rinse.

19. A method of treating a skin condition and/or ailment, including:
applying the topical apple product of claim 1 to a portion of skin condition repair skin dryness, irritation, rash, and/or melanoma.

20. The method of claim 19, wherein the apple material has a Polyphenols/Total Antioxidant Activity of ≧3.00%.
